(19) United States
(12) Patent Application Publication
(10) Pub. No.: US 2025/0311740 A1
(43) Pub. Date: Oct. 9, 2025

(54) High-Throughput Methods of Synthesizing Biofunctional Microparticles and Related Compositions

(71) Applicant: McMaster University, Hamilton (CA)

(72) Inventors: Lei Tian, Hamilton (CA); Zeinab Hosseinidoust, Dundas (CA)

(21) Appl. No.: 19/107,908

(22) PCT Filed: Aug. 30, 2023
(86) PCT No.: PCT/CA2023/051149

§ 371 Date: Feb. 28, 2025

Related U.S. Application Data
(60) Provisional application No. 63/402,093, filed on Aug. 30, 2022.

(51) Int. Cl. A23B 2/783; C12N 7/00

(52) U.S. Cl. CPC A23B 2/783; C12N 7/00; C12N 2795/14131; C12N 2795/14151

(57) ABSTRACT

Described herein is a high-throughput method of synthesizing biofunctional microparticles. In aspects, the method comprises casting biofunctional microparticle precursors onto a microporous template to form microparticles, wherein the template comprises a removable film; and removing the film to liberate the microparticles. Also described herein is a sprayable microgel and related methods.

### FIELD

The present disclosure relates to microparticles, and in particular, to microparticle synthesis and sprayable microparticle compositions and related uses thereof.

### BACKGROUND

Bacteriophages, also known as phages, are natural bacterial predators and their job in nature is to keep bacterial populations in check^{1,2}. Phages infect bacteria in a highly targeted manner—some are able to identify and kill a single strain of bacteria in a heterogeneous population. It follows that when used for biocontrol in environments with pre-existing commensal bacterial populations, such as certain food products or applications in agriculture, farming, or human therapeutic use, phages are less likely to disturb the delicate balance of such communities while still being able to eliminate harmful bacteria³. Foodborne diseases result in hundreds of thousands of deaths each year, almost a third of whom are young children⁴. Phage products have been approved by the US Food and Drug Administration for control of dangerous bacterial contaminants such as Escherichia coli, Salmonella, or Listeria in food products^{5,6}. The use of phage for food safety has the distinct advantage that, unlike most antimicrobials, it will not impact the taste, texture, and nutritional quality of the food, and can be safely applied to decontaminate food products from farm to market to consumer plates^{7,8}. However, widespread use is still limited. This is at least partly due to challenges in delivery and stability, which in turn limit the efficacy of the phage products⁹.

Nature makes phages in a variety of shapes and sizes¹⁰. Phages are, in essence, proteinous nanoparticles that encase a genome, enabling the propagation of wild-type or genetically modified virions into a suspension of identical and monodisperse nanoparticles¹¹. In addition, the phage surface chemistry can be customized with atomic precision via genetic engineering or chemical conjugation, making phage virions a powerful building block for multifunctional antimicrobial material¹²⁻¹⁴.

Previously reported was a simple chemistry that was effective at crosslinking filamentous phage, yielding bulk soft material displaying the basic properties of a hydrogel¹⁵⁻¹⁷. Compared to polymeric microgels such as poly(N-isopropylacrylamide)¹⁸ and poly(ethylene glycol)¹⁹, phage microgels remain unexplored, partly because of challenges in manufacturing such microgels. Common microgel preparation methods such as microfluidics²⁰ or the emulsion method²¹ are not suitable for microgels encapsulated with or made from heat/solvent sensitive chemicals or biomolecules (such as proteins and viruses) that must retain their bioactivity through the preparation process.

Microgels offer major distinct advantages over bulk material. Namely, they have larger surface areas and thus more contact points for phage with contaminating bacteria as well as enhanced flow properties in suspensions, allowing for delivery via spray or injection, all of which make them a more versatile option for biocontrol in environmental, food, and medical applications²²⁻²⁴. Packing phages into soft, hydrated material further has the advantage of preservation against desiccation and harsh environments^{25,26}. The hydrated structure of microgels offers the advantage of preserving desiccation-sensitive biomolecules. There is thus a need for novel phage gels through the development of a high throughput manufacturing method that enables the generation of microgels that are made up entirely of viral nanoparticles while also preserving the bioactivity of these phage building blocks in the process.

The background herein is included solely to explain the context of the disclosure. This is not to be taken as an admission that any of the material referred to was published, known, or part of the common general knowledge as of the priority date.

### SUMMARY

The present application discloses a biomolecule-friendly, high-throughput method to synthesize sprayable phage microgels that serve as a high-load delivery vehicle for protein and strong virulent phages to control the growth of microorganisms in food products and other biocontrol scenarios. Phage microgels are also described.

In accordance with an aspect, there is provided a high-throughput method of synthesizing sprayable phage microgels, comprising: a) suspending phage microgels to a sprayable fluid composition; b) applying an effective amount of the sprayable fluid composition to a surface, wherein the sprayable fluid composition comprises phage microgels, and a liquid medium.

In some embodiments, the microgels are synthesized using a template.

In some embodiments, the template is microporous.

In some embodiments, the microgels are synthesized using microfluidics or emulsion methods.

In some embodiments, the microgels comprise a high density of phage.

In some embodiments, the microgels are prepared as a microgel suspension.

In some embodiments, the microgel suspension comprises phage microgels and a liquid.

In some embodiments, the liquid comprises water, buffer solution, phosphate buffer saline (PBS), beverages, medicine, or combinations thereof.

In some embodiments, the microgels are prepared in a state that can be delivered as a spray.

In some embodiments, the microgels comprises one type or multiple types of phage.

In some embodiments, the phage comprises Escherichia coli bacteriophages, M13, HER262, T7 bacteriophages, or combinations thereof.

In some embodiments, the phage comprises unmodified bacteriophages, chemically-modified bacteriophages, genetically-modified bacteriophages, or combinations thereof.

In some embodiments, the phages are self-crosslinked, or crosslinked with one or more crosslinkers.

In some embodiments, the one or more crosslinkers comprises glutaraldehyde, 1-Ethyl-3-(3-dimethylaminopropyl) carbodiimide, or combinations thereof.

In some embodiments, the microgels comprise a protein or other molecule with functional groups able to react with crosslinkers.

In some embodiments, the protein comprises bovine serum albumin (BSA).

In some embodiments, the protein or other molecule is used to preserve phage bioactivity.

In some embodiments, the microgels are applied using a spray applicator.

In some embodiments, the phage microgels are used as microgel biosensors and bioassays.

In some embodiments, the sprayable phage microgels are used for biocontrol such as, but not limited to environmental, food, agricultural, pharmaceutical, medical, nutraceutical, textile, cosmetic, or industrial applications.

In some embodiments, the sprayable phage microgels are sprayed onto food products or food contact environments such as, but not limited to food packaging facilities.

In accordance with an aspect, there is provided a sprayable phage microgel effective in controlling the presence of microorganisms on a surface comprising: the sprayable phage microgel, and a spray applicator.

In accordance with an aspect, there is provided a phage-exclusive cross-linked microgel.

In some embodiments, the phage is cross-linked via amide bonds between two phages.

In some embodiments, the phage is cross-linked with EDC.

In some embodiments, the microgel has high porosity.

In some embodiments, the microgel has a homogenous nanofibrous texture along the same orientation.

In some embodiments, the microgel is loaded with protein, phage, small molecules, or combinations thereof.

In some embodiments, the microgel is loaded with virulent phage.

In accordance with an aspect, there is provided a virulent phage-embedded phage microgel.

In some embodiments, the microgel further comprises BSA.

In some embodiments, the virulent phage is HER262 or T7.

In accordance with an aspect, there is provided a phage microgel comprising a size distribution of from about 23±6 μm to about 33±6 μm.

In accordance with an aspect, there is provided food packaging, food spray, or a household cleaning product comprising the microgel described herein.

In accordance with an aspect, there is provided a sprayable microgel, where the microgel is the microgel as defined herein.

In accordance with an aspect, there is provided a high-throughput method of synthesizing sprayable phage microgels, the method comprising:
casting a composition comprising phage and a cross-linker on a template film comprising micropores and allowing gelation of the phage,
peeling off the film, thereby liberating microgels from within the pores, and
suspending the microgels in a sprayable liquid medium.

In accordance with an aspect, there is provided a high-throughput method of synthesizing biofunctional microparticles, the method comprising: casting biofunctional microparticle precursors onto a microporous template to form microparticles, wherein the template comprises a removable film; and removing the film to liberate the microparticles.

In some embodiments, the template and/or film comprises polystyrene, polyvinyl chloride, polycarbonate, polyimide, polyvinyl chloride, polyvinyl butyral, or combinations thereof.

In some embodiments, the microporous template is prepared by the breath figure method, micro-templating, or photolithography.

In some embodiments, the size range of micropores and resulting microparticles is between about 0.1 μm and about 999.9 μm.

In some embodiments, the micropores and resulting microparticles are spherical, semi-spherical, cylindrical, or spindle-shaped.

In some embodiments, the biofunctional microparticle precursors comprise biological materials, synthetic materials, or combinations thereof.

In some embodiments, the biological materials comprise proteins, peptides, enzymes, antibodies, nucleic acids, viruses, phages, prokaryotic cells, eukaryotic cells, or combinations thereof.

In some embodiments, the phages comprise unmodified phages (wild type), chemically-modified phages, genetically-modified phages, or combinations thereof.

In some embodiments, the biofunctional microparticle precursors comprise additives.

In some embodiments, the additives comprise chemical or physical crosslinkers, nanoparticles, phages, antibiotics, proteins, peptides, nucleic acids, viruses, polymers, or combinations thereof.

In some embodiments, the biofunctional microparticle precursors are self-crosslinked or crosslinked with one or more physical or chemical crosslinkers.

In some embodiments, the crosslinker comprises glutaraldehyde, 1-Ethyl-3-(3-dimethylaminopropyl) carbodiimide, gold nanoparticles, polymeric crosslinkers, or combinations thereof.

In some embodiments, the microparticles are prepared in a suspension, solid patch, or powder.

In some embodiments, the microparticle suspension comprises microgels.

In some embodiments, the microgels comprise phage microgels and a fluid.

In some embodiments, the fluid comprises water, a buffer solution, phosphate buffered saline (PBS), a beverage, a medicine, or combinations thereof.

In some embodiments, the microparticle suspension is for delivery in a spray, a suspension, by nebulization, or by injection.

In accordance with an aspect, there are provided biofunctional microparticles made by the methods described herein.

In accordance with an aspect, there is provided a phage-built microgel made by the methods described herein.

In accordance with an aspect, there is provided a sprayable microgel made by the methods described herein.

In accordance with an aspect, there is provided a method of controlling bacteria, the method comprising applying the spray of claim 20 to a bacteria-susceptible surface.

In some embodiments, the surface comprises a food product, food packaging, or food contact environment.

In some embodiments, the food contact environment comprises a food packaging facility.

In accordance with an aspect, there is provided a use of the sprayable microgel described herein for controlling bacteria in environmental, food chain, agricultural, pharmaceutical, medical, nutraceutical, textile, cosmetic, household, healthcare applications, or combinations thereof.

Other features and advantages of the present disclosure will become apparent from the following detailed description. It should be understood, however, that the detailed description and the specific examples, while indicating embodiments of the disclosure, are given by way of illustration only and the scope of the claims should not be limited by these embodiments, but should be given the broadest interpretation consistent with the description as a whole.

### DRAWINGS

Certain embodiments of the disclosure will now be described in greater detail with reference to the attached drawings in which:

FIG. 1 shows the preparation of bacteriophage microgels using honeycomb film as template in exemplary embodiments of the disclosure: a, Crosslinking reactions of M13 bacteriophages with GA and EDC respectively; b, Schematic image of preparing phage microgels: Honeycomb film is plasma-coated to increase hydrophilicity; Phage and crosslinker mixture solution is cast on the film and placed in the vacuum for 10 mins; The film is placed in a humid environment in 4° C. for 2 days; The top layer of the film is peeled off using adhesive tape; The phage microgels are isolated after removing the film; c, Surface and cross-section image of the polystyrene honeycomb film surface (scale bar 20 um). Insert: photo of the round polystyrene honeycomb film (scale bar 1 cm); d, Surface and cross-section SEM images of M13 crosslinked by GA inside the pores of honeycomb film (scale bar 20 μm); e, A flexible honeycomb film containing phage microgel array. Photos of an independent composite film tailored into a 1 cm² square, further showing the film can easily be bent by hand; f, SEM images of phage microgel array left on a peeled honeycomb film (scale bar 10 μm) and zoom-in SEM image of a single phage microgel in this array (scale bar 5 μm); g, SEM images of the permeable pore network on adhesive tape after peeling (left) and the honeycomb film after peeling and sonication (right). Scale bar 20 μm; h, SEM images of the isolated phage microgels (scale bar 500 μm) and a single phage microgel made with M13 phages and GA (scale bar 5 μm); i, SEM images of a phage microgel made with M13 crosslinked with EDC (left) and GA+BSA (right). Scale bar 10 μm; j, Size distribution of the template pores (n=84) and the phage microgels prepared with GA (n=58, 57), EDC (n=45, 53) and BSA (n=56, 54) at hydrated and dried status. (Violin plot lines indicate 25^{th}, 50^{th}, 75^{th} percentile); k, Pore density of the template (n=7) and the produced microgel count from every square centimeter of the template (n=6). Box plots show minimum to maximum (whiskers), 25-75% (box), median (band inside) with all data points.

FIG. 2 shows the crosslinking reactions between M13 phage and the other formations of GA in aqueous solution in exemplary embodiments of the disclosure: a, Two M13 phages react with cyclic hemiacetal and incorporate; b, Two M13 phages react with cyclic hemiacetal and incorporate.

FIG. 3 shows the fluorescence profile of M13 phage microgels and fluorescence profile of the template before and after the microgel formation in exemplary embodiments of the disclosure: a-c, Fluorescent images of three types of phage microgels made of 5×10¹³ PFU/mL of M13 phage with a, 0.1 M GA, b, 0.1 M EDC, and c, 2% BSA +0.1 M GA. Scale bar: 100 um; d, Quantified fluorescent intensity of 3 types phage microgels under four different channels. Box plots show minimum to maximum (whiskers), 25-75% (box), median (band inside) with all data points (n=4-5); e, FTIR spectra of phage microgels; f-h, fluorescent images of honeycomb template filled with the mixture solution corresponding to a-c. Scale bar: 500 μm; i-k, fluorescent images of corresponding honeycomb films after the gelation of phage solution inside. Scale bar: 500 μm. Different fluorescent channels are: 1, bright field; 2, film excited at 340 nm and imaged using a λ=435 nm optical filter (blue channel); 3, film excited at 465 nm and imaged using a λ=515 nm optical filter (green channel); 4, film excited at 528 nm and imaged using a λ=590 nm optical filter (orange channel) and 5, film excited at 625 nm and imaged using a λ=670 nm optical filter (red channel).

FIG. 4 shows the isolation of phage microgels in exemplary embodiments of the disclosure: a, Detailed Schematic image of the peeling process: the top half of the pores was removed and the microgels inside the film were exposed on the bottom film layer without damage; b, SEM image of the edge of peeling area of the honeycomb film: The peeled area is on the top left presenting shallow pores and the unpeeled area is on the bottom right showing deep spherical pores; c, large-scale SEM image of the adhesive tape after peeling. Scale bar: 50 μm; d, large-scale SEM image of the honeycomb film after peeling; Scale bar: 50 μm.

FIG. 5 shows the bioactivity of 1×10⁶ PFU/mL of free HER262 phages and M13+HER262+BSA+GA hybrid phage microgels (before and after air-drying for 1 hr) on E. coli O157:H7 lawn in exemplary embodiments of the disclosure.

FIG. 6 shows the evaluation of microgel preparation efficiency in exemplary embodiments of the disclosure: a, Image of the honeycomb film with microgels inside with rule for film area measurement; b, Bright field image of 5 μl of phage microgel suspension for microgel count. Scale bar: 1 mm; c, Zoom-in bright field image of b showing free-stand microgels. Scale bar: 100 μm.

FIG. 7 shows the aligned nanofibrous texture of phage microgels in exemplary embodiments of the disclosure: a, SEM image of non-crosslinked M13 phage nanofibers and schematic image of a nanofilamentous M13 phage showing high aspect ratio; b, Schematic image of a hydrated phage microgel composed of crosslinked filamentous M13 phages turning into a phage acrogel microparticle by critical point drying, showing a nanofibrous texture; c, SEM images of a M13 phage microgel crosslinked by GA and the highly-aligned nanofibrous texture on the microgel surface; d, SEM images of the orderly-aligned nanofibrous texture on a M13 phage microgel crosslinked by EDC; e, SEM images of the nanofibrous texture on a M13+BSA microgel crosslinked by GA.

FIG. 8 shows the nanostructure of the BSA microgel in exemplary embodiments of the disclosure: a, SEM image of BSA microgels crosslinked by GA in peeled honeycomb template; b, Nanostructure on the surface of a BSA microgel; c, Zoom-in image of image b.

FIG. 9 shows antimicrobial activity of hybrid phage microgels in exemplary embodiments of the disclosure; a, Schematic image of a phage microgel where the M13 g3p is binding to the tip of the F pilus on the host E. coli. Box on the bottom right: Comparison of the shapes of phage M13 (filamentous), HER262 (long tailed), T7 (short tailed); b, Photos of the HER262-embedded hybrid phage microgels patch arrays forming lysis zones on the lawn of both E. coli ER2738 and E. coli O157:H7; c, HER262-embedded hybrid phage microgels sprayed on the lawn of both E. coli ER2738 and O157:H7, showing clearing zones on both lawns; d, Titer count of E. coli O157:H7 incubated in PBS after 9 hrs at different initial concentration with (n=5) and without (n=3) HER262 microgels; e, Kill curves for E. coli O157:H7 suspension, incubated in TSB for 9 hrs at different initial concentration with and without HER262 microgels (n=3); f, Final titer count of the E. coli O157:H7 incubated in TSB with (n=5) and without (n=3) HER262 microgels after 9 hrs in part e; g, Left: Schematic image of microgel sprays decontaminating multidrug-resistant E. coli O157:H7 in lettuce. Right: pictures of lettuce and meat. White boxes indicate where the lettuce and meat were cut into small pieces; h, Left: pictures of wrapped artificially contaminated lettuces sprayed with water and microgels, respectively at 0 and 9 hours. Right: bacterial titer count of the collected bacterial suspension from contaminated meat (n=12); i, Left: pictures of wrapped artificially contaminated meats sprayed with water and microgels respectively at 0 and 9 hours. Right: bacterial titer count of the collected bacterial solution from the contaminated lettuces (n=12). (Error bars indicate standard deviation. Box plots show minimum to maximum (whiskers), 25-75% (box), median (band inside) with all data points. ****P<0.0001. Statistical significance in all panels is derived from one-way analysis of variance (ANOVA). Schematics created with BioRender.com.)

FIG. 10 shows the specific infectivity and desiccative sensitivity of phage M13, HER262 and T7 in exemplary embodiments of the disclosure: a, Infectivity of phage M13, HER262 and T7 to four types of bacterial strains; b, Titer count of phage M13, 8 and T7 before (n=3) and after desiccation (n=6) for 1 h. (Error bars indicate standard deviation, ***P<0.001, ****P<0.0001).

FIG. 11 shows the antimicrobial property of different M13 phage microgels in exemplary embodiments of the disclosure: a, Photo of the sprayer containing phage microgel suspension; b, Patches and sprayed microgels on the lawn of E. coli ER2738; c, Left: Growth curve of E. coli ER2738 in LB solution with and without adding microgels. Box on the top left: Schematic of the phage component in these microgels. Middle: Titer count of E. coli ER2738 after incubating 12 hours in LB with and without microgels; Right: Titer count of phage M13 in the E. coli ER2738 LB solution with microgels at 0 and 12 h. (Error bars indicate standard deviation, n=3, *P<0.05, ****P<0.0001).

FIG. 12 shows the nanostructure of M13+HER262+BSA+GA hybrid phage microgels in exemplary embodiments of the disclosure: a, SEM images of a M13+HER262+BSA+GA microgel; b-d, SEM images of nanostructure on the surface of the microgel.

FIG. 13 shows M13+HER262+BSA+GA hybrid phage microgels incubating with other E. coli strains in exemplary embodiments of the disclosure: a, Optical density growth of E. coli ER2738 (106 CFU/mL) incubated in TSB during 9 hours with and without phage HER262 microgels. Box on the bottom right: Schematic of the 2 phage components in these microgels; b, Optical density growth of E. coli BL21 (106 CFU/mL) incubated in TSB during 9 hours with and without phage HER262 microgels; c, Final titer count of the E. coli ER2738 and BL21 incubated in TSB after 9 hours in figure a-b. (Error bars indicate standard deviation, n=3).

FIG. 14 shows the antimicrobial property of M13+T7+BSA+GA hybrid phage microgels in exemplary embodiments of the disclosure: a, Schematic of the 2 phage components in these microgels; b, Photos of the hybrid T7 microgels in the patches and spray on the lawn of E. coli BL21; c, Titer count of E. coli BL21 incubated in PBS after 9 hours at different initial concentration with and without T7 microgels; d, Left: Optical density growth of E. coli ER2738 (10⁶ CFU/mL) incubated in TSB during 9 hours with and without T7 microgels. Right: Compact bacterial lawn and sporadic colonies formed by the bacterial solution incubated without and with microgels respectively; e, Titer count of the BL21 in TSB after 9 hours incubated with and without microgels. (Error bars indicate standard deviation, n=3, *P<0.05, **P<0.01, ***P<0.001).

### DETAILED DESCRIPTION

### I. Definitions

Unless otherwise indicated, the definitions and embodiments described in this and other sections are intended to be applicable to all embodiments and aspects of the present disclosure herein described for which they are suitable as would be understood by a person skilled in the art. It is also to be understood that the terminology used herein is for the purpose of describing particular aspects only and is not intended to be limiting.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies. In addition, all ranges given herein include the end of the ranges and also any intermediate range points, whether explicitly stated or not.

As used in this disclosure, the singular forms “a”, “an” and “the” include plural references unless the content clearly dictates otherwise.

In embodiments comprising an “additional” or “second” component, the second component as used herein is chemically different from the other components or first component. A “third” component is different from the other, first, and second components, and further enumerated or “additional” components are similarly different.

The term “and/or” as used herein means that the listed items are present, or used, individually or in combination. In effect, this term means that “at least one of” or “one or more” of the listed items is used or present.

The abbreviation, “e.g.” is derived from the Latin exempli gratia and is used herein to indicate a non-limiting example. Thus, the abbreviation “e.g.” is synonymous with the term “for example.” The word “or” is intended to include “and” unless the context clearly indicates otherwise.

The term “hydrogel”, as used herein refers to a material that exhibits the ability to swell and retain a significant fraction of water within its structure, without dissolving in water. It will be understood that conventional hydrogels typically comprise a water-swellable polymeric matrix, consisting of a three-dimensional network of hydrogel polymers (e.g., hydrophilic polymers, hydrophobic polymers, blends thereof, such as poly (ethylene glycol), collagen, gelatin, dextran, elastin, alginate, hyaluronic acid, poly (vinyl alcohol), derivatives thereof, and combinations thereof) held together by covalent or non-covalent crosslinks. While the hydrogels described herein may comprise such polymers as additional components, the hydrogels described herein may comprise only crosslinked bacteriophages. It has now been found that crosslinked bacteriophages result in a hydrogel composition that can absorb a substantial amount of water (e.g., 50%, 60% 70%, 80%, 90%, 95%, 96%, 97%, 98%, 99% or greater than 99% per unit of non-water molecule) to form a water-swellable gel.

The term “crosslinked”, as used herein refers to a composition containing intramolecular and/or intermolecular crosslinks, whether arising through covalent or noncovalent bonding, and may be direct or include a cross-linker. “Non-covalent” bonding includes both hydrogen bonding and electrostatic (ionic) bonding.

The term “self-healing”, as used herein refers to a material that when broken or cut, has the ability to substantially return substantially to an initial state or condition prior to being broken or cut to retain material integrity. This healing process can be aided by stimuli, including but not limited to, electrolytes, ions, proteins and/or peptides, change in temperature and/or pH, or applying an electric or a magnetic field.

It will be understood that any component defined herein as being included may be explicitly excluded by way of proviso or negative limitation, such as any specific compounds or method steps, whether implicitly or explicitly defined herein.

### II. Methods and Compositions

Described herein are methods for synthesizing biofunctional microparticles. In aspects, the methods are high-throughput. Typically, the methods comprise a casting process, whereby precursors for the biofunctional microparticles are cast onto a microporous template comprising a plurality of micropores. The template typically comprises a removable film, so that the pores are open-ended at one end and closed by the film at the other end. In this way, casting of the biofunctional microparticle precursors leads to the microparticles being cast on the film at the bottom of the pores. Once the microparticles are formed, the film can be removed, for example, peeled away from the template, in order to liberate the particles. The particles can be liberated from the film by many different methods but are typically placed into a fluid medium to release the microparticles from the film.

The template and/or film can be made of many different materials that are typically acceptable for such applications. For example, typically, the template and/or film is made from or comprises at least in part polystyrene, polyvinyl chloride, polycarbonate, polyimide, polyvinyl chloride, polyvinyl butyral, or combinations thereof.

Similarly, the microporous template may be prepared by any number of methods. Typically, it is prepared by the breath figure method, micro-templating, or photolithography.

The micropores and resulting microparticles will be understood to have a size in the micron range, such as from about 0.1 μm to about 999.9 μm. Typically, the pores and microparticles have a size of from about 0.1, about 1, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 60, about 70, about 80, about 90, about 100, about 200, about 300, about 400, about 500, about 600, about 700, about 800, or about 900 microns to about 1, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 60, about 70, about 80, about 90, about 100, about 200, about 300, about 400, about 500, about 600, about 700, about 800, about 900 microns, or less than 1 mm. Typically the size range is from about 20 to about 40 microns. These pore sizes will be understood to be distributions and therefore not all pores are identical in size, therefore these values may be +/− up to about 6 or 7 microns.

The pores can be of any desired shape. For convenience and most applications, typically the micropores and resulting microparticles are spherical, semi-spherical, cylindrical, or spindle-shaped. Combinations of shapes can also be employed.

The biofunctional microparticles can be made from any variety of precursors depending on the desired final product and use. For example, the precursors may comprise biological materials, synthetic materials, or combinations thereof. Typically, the biological materials comprise proteins, peptides, enzymes, antibodies, nucleic acids, viruses, phages, prokaryotic cells, eukaryotic cells, or combinations thereof. In specific aspects, as exemplified herein, the biological materials comprise phages. The phages will be understood to comprise unmodified phages (wild type), chemically-modified phages, genetically-modified phages, or combinations thereof.

The biofunctional microparticle precursors may further comprise additives, which can be selected depending on the desired end use and product. For example, the additives typically comprise chemical or physical crosslinkers, nanoparticles, phages, antibiotics, proteins, peptides, nucleic acids, viruses, polymers, or combinations thereof.

In typical aspects, the biofunctional microparticle precursors are self-crosslinked or crosslinked with one or more physical or chemical crosslinkers. Thus, in some aspects, the method involves waiting a period of time for the precursors to crosslink or otherwise gel or set. The crosslinker typically comprises glutaraldehyde, 1-Ethyl-3-(3-dimethylaminopropyl) carbodiimide, gold nanoparticles, polymeric crosslinkers, or combinations thereof.

The microparticles can be prepared in different compositions. For example, they can be provided as a suspension, a solid, such as a solid patch, or a powder. Typically, when the microparticles are provided as a suspension, the microparticles are in the form of microgels. Thus, in some aspects, a suspension comprising microgels is provided. In certain typical aspects, the microgels comprise phage microgels and a fluid. Any fluid can be used, however, typically the fluid comprises water, a buffer solution, phosphate buffered saline (PBS), a beverage, a medicine, or combinations thereof.

The microparticle suspension can further be provided in different compositions, For example, typically the suspension is for delivery in a spray, however, it may also be provided in a suspension, by nebulization, or by injection, for example.

Also provided are biofunctional microparticles made by the methods described herein. More specifically, provided are is a phage-built microgel made by the methods described herein. Also provided is a sprayable microgel, such as a sprayable phage-built microgel made by the methods described herein.

The sprayable microgel described herein is useful in methods of controlling bacteria, by simply applying the sprayable microgel to a bacteria-susceptible surface. Any surface can be sprayed, for example, inert objects such as counters, floors, door handles, medical equipment, and so on. Further, objects such as skin may be sprayed if all components in the composition are considered safe for human applications. Typically, the bacteria-susceptible surface comprises a food product, such as meat, vegetables, or fruit, for example, food packaging, such as food trays or plastic wrap, for example, or food contact environments, such as surfaces found in a food packaging facility.

Further, the sprayable microgel described herein may be used for controlling bacteria in environmental, food chain, agricultural, pharmaceutical, medical, nutraceutical, textile, cosmetic, household, healthcare applications, or combinations thereof.

### EXAMPLES

The following non-limiting examples are illustrative of the present disclosure:

### Example 1

### Methods

Preparing polystyrene honeycomb film. Honeycomb films were prepared using the breath figure method, as described in detail elsewhere^{35,44}. This high-throughput method does not require any large equipment or any premade microparticles as templates. Briefly, 600 μL of 5 wt % of polystyrene (Mw=650 000, Millipore Sigma) in chloroform was cast and spread circularly on a clean glass slide in a humid chamber (˜55% relative humidity monitored by a humidity sensor). The chamber was sealed immediately after adding the polystyrene solution to maintain humidity. After 20 mins, the polystyrene solution had solidified, forming a white film on the glass slide. The slides were then taken out of the chamber. After 1 h, the honeycomb film was easily peeled off and stored at room temperature.

Phage propagation, purification and concentrating. M13 bacteriophage was propagated using its host: Escherichia coli strain K12 ER2738 (New England Biolabs Ltd., E4104S). A pre-culture of E. coli was prepared in LB-Miller broth and placed in a shaking incubator overnight set to 180 rpm and 37° C. The following day, 2.5 mL of the pre-culture was added to 250 mL of LB broth in a baffled flask. Subsequently, a 10 μL aliquot of M13 phage (10¹² PFU/mL) was added to the flask to initiate the propagation. The flask was incubated in a shaking incubator set to 180 rpm and 37° C. for 5 hrs. 50 mL aliquots of propagated phage solution were then centrifuged at 7000×g for 15 mins. The resulting bacteria pellets were discarded, and the phage-containing supernatant was stored at 4° C.

The purification of the propagated M13 phage supernatant was achieved through an aqueous two-phase polyethylene glycol (PEG) precipitation protocol followed by an ultracentrifugal filtration, as described previously by Sambrook⁴⁵. A 20 (w/v) % PEG solution was aseptically prepared and supplemented with 2.5 M NaCl solution. The sterile PEG solution was added in a 1:6 ratio to the propagated phage supernatant and incubated in a fridge overnight at 4° C. Subsequently, the incubated PEG-phage solutions were centrifuged at 4° C. and 5000×g for 45 mins to obtain pelleted phage. The resulting phage was then resuspended in 5 mL of sterilized water and incubated overnight on a roller at 4° C. The resuspended phage was then centrifuged at 5000×g for 15 mins to remove the remaining bacterial contaminants. The described PEG/NaCl purification procedure was subsequently repeated a second time to ensure all contaminants were removed. The resulting PEG-purified phage solution was then filtered through centrifugal filters (MWCO 100 KDa and 30 KDa, Millipore Sigma, Ultra-15) to remove excess water. The final concentration was titered using plaque assay method⁴⁶.

Phage microgel preparation. A polystyrene honeycomb film was used as the template to prepare the phage microgels. The film was initially plasma-coated with O₂ for 5 mins and then covered with 100 μL of the mixture of M13: 5×10¹³ PFU/mL with GA or EDC: 0.1 M. The film was subsequently placed inside a desiccator connected to a vacuum pump. The pump was turned on for 5 mins to create a low-pressure environment which helped the phage solution fill inside the micropores. The film was then taken out and transferred into a sealed humid container at 4° C. for 1 day.

After 2 days, a glass slide was used to remove the excess phage hydrogel on the template surface. After this cleaning step, a piece of transparent adhesive tape was adhered to the template film surface and then peeled off to remove the top layer of the template. Then the template film was immersed in 1 mL of sterilized water or PBS and sonicated for 10 mins. After the sonication, the film was taken out and discarded. The microgels were suspended in water and stored at 4° C. for further experiments.

Scanning Electron Microscopy. Samples were pre-treated using the critical point drying method to dehydrate the microgels without disturbing their surface nanostructures. Samples were processed through an ethanol gradient treatment and then placed in a Leica critical point dryer (EM CPD300) for 3.5 hrs.

Two types of Scanning Electron Microscopy (SEM) were used to image the templates and microgels. TESCAN VEGA-II LSU SEM was used to image these samples, where 10 nm layers of gold were coated onto the samples in advance. A field emission scanning electron microscope (FEI Magellan 400) was used to image the nanostructure on the surface of the microgels, where 3 nm layers of Pt were coated onto the samples in advance.

Inverted Fluorescence Microscopy. An inverted microscope (Nikon Eclipse Ti2 inverted microscope) was used to take bright field and fluorescent images of the microgels and their templates. Four different optical filter sets (blue channel: ex/em=340/435 nm; green channel: ex/em=465/515 nm; orange channel: ex/em=528/590 nm; red channel: ex/em=625/670 nm) were used for fluorescence imaging. The excitation filter was positioned in front of the LED light source, and the image was captured using the emission filter attached to the camera. The intensity of the light source and the exposure time were consistent.

Size measurement of template pores and microgels. An inverted microscope (Nikon Eclipse Ti2) was used to image the template pores and microgels. The size of pore and microgels were measured using the NIS-Elements AR software. The diameter of a template pore was defined as the diameter of the spherical hole instead of the surface pore, as spherical holes determine the microgel size and can easily be measured using emission light mode. For each sample, 9 images from 3 samples were captured randomly, and all pores/particles were measured to collect the diameter data.

Microgel preparation efficiency. The pore density of the honeycomb film was defined as the pore count divided by the film area. 9 images of 3 honeycomb films were taken using a Nikon Eclipse Ti2 inverted microscope at 40×. All pores in the frames were manually counted and the frame areas were measured using the software NIS-Elements AR.

The microgels isolated from the templates were collected in 1 mL of Millipore water. To count the number of microgels in the 1 mL suspension, a 5 μL sample was drop-cast on a glass slide and a large image covering the entire droplet was taken using an inverted microscope. The number of microgels in this droplet n_{5 μL} was then manually counted, and the total amount of microgel was calculated using this equation: n_{1 mL}=n_{5 μL}×200. For each type of microgel, the procedure was repeated at least four times.

Fourier transform infrared (FTIR) spectra. FTIR spectra of the phage microgels were represented under by phage hydrogel bulks made with materials exactly same as corresponding microgels. Phage hydrogels were pre-dehydrated, placed in the FT-IR Spectrometer (Nicolet 6700, Thermo Fisher Scientific) and the spectra were collected in the range of 4000-500 cm⁻¹ using 128 scans at a resolution of 4 cm⁻¹.

Desiccation sensitivity test for phage. A 10 μL drop of phage suspension (M13, HER262 and T7, ˜10¹⁰ CFU/mL) was added on a clean, uncovered glass slide at room temperature. The suspension was dried in 10 mins and continued to desiccate afterwards. After 1 hr, 10 μL of sterile PBS was used to resuspend the phage. The final and original concentrations of phages were titered through full plate plaque assays. The procedure was repeated in triplicate for each type of phage. Phage HER262 was purchased from the Félix d'Hérelle Reference Center for bacterial viruses of the Laval University, and T7 was from Leibniz Institute DSMZ-German Collection of Microorganisms and Cell Cultures.

Antimicrobial test of phage microgel patches on bacterial lawn. In this experiment, the phage microgels were not isolated from the template. Instead, the composite films were regarded as flexible antimicrobial patches, representing an ordered monolayer of phage microgels. After microgel gelation, the composite patches were washed in sterile water for 20 mins to remove free phages.

Luria-Bertani (LB) agar plates were prepared by suspending LB powder (25 g/L, Fisher Scientific) in sterile water and supplemented with agar (1.5% w/v, 15 g/L, Fischer Scientific) and dispensed into petri dishes (100 mm×15 mm, sterile, polystyrene, Fisher Scientific) using a sterile serological pipette. Soft agar overlays were prepared by boiling sterile water supplemented with LB Broth powder (25 g/L) and agar (0.6% w/v, 6 g/L). A 3 mL aliquot of boiled media was dispensed into glass test tubes. Test tubes were then autoclaved to ensure sterility.

Lawns of bacterial overlay were prepared by suspending 100 μL of bacterial suspensions (E. coli ER2738, O157:H7, or BL21) in 3 mL of liquefied soft LB-agar, which was vortexed and poured on LB agar plates. After the soft agar was solidified, the washed patches were gently placed on top of the bacterial lawn. The double layer plates were incubated in a stationary incubator (37° C., VWR International Co.) overnight and subsequently imaged.

Antimicrobial test for phage microgel sprays on bacterial lawn. 1 mL of the fresh-made phage microgel suspension was transferred into a sprayer. Phage microgel solution was then sprayed on bacterial lawns (prepared as previously described). The double layer agar plates were then incubated in a stationary incubator (37° C.) overnight and subsequently imaged.

Antimicrobial test for phage microgel suspensions. Two different media, Tryptic Soy Broth (TSB) and nutrient-deficient PBS, were prepared to evaluate the bactericidal ability of phage microgels in different liquid environments.

Nutrient-rich environment. Overnight bacterial cultures (E. coli O157:H7 or BL21, ˜10⁹ CFU/mL) grown in TSB were diluted to 1:10, 1:100 and 1:1000 in fresh TSB media. For each dilution and the original overnight culture, 10 replicates of 200 μL bacterial solution were added to a sterile 96-well plate. A 10 μL aliquot of the phage microgel suspension was then added to each of the first three replicates as the sample group (labelled “With microgels”). A 10 μL drop of sterile PBS was added to the remaining three replicates as the control group (labelled “No microgels”). Subsequently, the 96-well plate was placed in a microplate reader (Synergy Neo2 Hybrid Multi-Mode Reader, 37° C., 180 rpm) to measure optical density at a wavelength of 600 nm (OD₆₀₀) every 20 mins for 9 hrs. Bacterial CFU counts of each replicate were obtained at the end point.

Nutrient-deficient environment. Overnight bacterial cultures (E. coli O157:H7 or BL21, ˜10⁹ CFU/mL) grown in TSB were diluted to 1:10, 1:/100, 1:1000 and 1:10000 in PBS. For each dilution and the original overnight culture, 10 replicates of 200 μL bacterial solution were added to a sterile 96-well plate. A 10 μL aliquot of the phage microgel suspension was then added to each of the first three replicates as the sample group (named “With microgels”). A 10 μL drop of sterile PBS was added to the remaining three replicates as the control group (named “No microgels”). Afterwards, the 96-well plate was placed in a shaking incubator (Thermo Scientific, 37° C., 180 rpm) for 9 hrs, and the bacterial titer count of each sample at the end point was calculated.

Food decontamination test of phage microgels. Lettuce (romaine heart) was purchased at the local supermarket and cut into 6 squares weighing 0.4±0.01 g. 4 samples were contaminated with E. coli O157:H7, reaching a contamination level of 10⁶ CFU/g. A 200 μL aliquot of the phage microgel suspension was then sprayed onto two contaminated leaves directly while the other two contaminated leaves were sprayed with sterile water. The remaining two leaves served as controls and were wrapped by food wraps without treatment. All 6 lettuce squares were wrapped and placed at room temperature for 9 hrs. The lettuce squares were then immersed in 10 mL of sterile PBS. Then, the samples were unwrapped and immersed in 4 mL of sterile PBS. This mixture was vortexed for 2 mins to dislodge bacteria and the titer was determined using standard colony count. MacConkey-Sorbitol ChromoSelect Agar (Millipore Sigma) plates were used for selective O157:H7 titer count⁴⁷ (Detection limit: 100 CFU/g, performed in triplicate for each sample in two independent experiments).

The decontamination test for beef steaks (Canadian beef, AAA Angus) followed a similar protocol. Beef steaks were cut into 6 cubes weighing 3±0.1 g. A 30 μL aliquot of E. coli O157:H7 (10⁸ CFU/mL) was added to 4 meat cubes to achieve a contamination level of 10⁶ CFU/g. A 200 μL aliquot of the phage microgel suspension was then sprayed onto 2 contaminated meat cubes directly while the other 2 contaminated cubes were sprayed with sterile Millipore water. The remaining 2 cubes served as controls and were wrapped by food wraps without treatment. The 6 meat cubes were placed at room temperature for 9 hrs. The samples were then unwrapped and immersed in 10 mL of sterile PBS. This mixture was vortexed for 2 mins to dislodge the bacteria and bacteria titer was determined using standard colony counts (Detection limit: 34 CFU/mL, performed in duplicate for each sample in two independent experiments).

### Results and Discussion

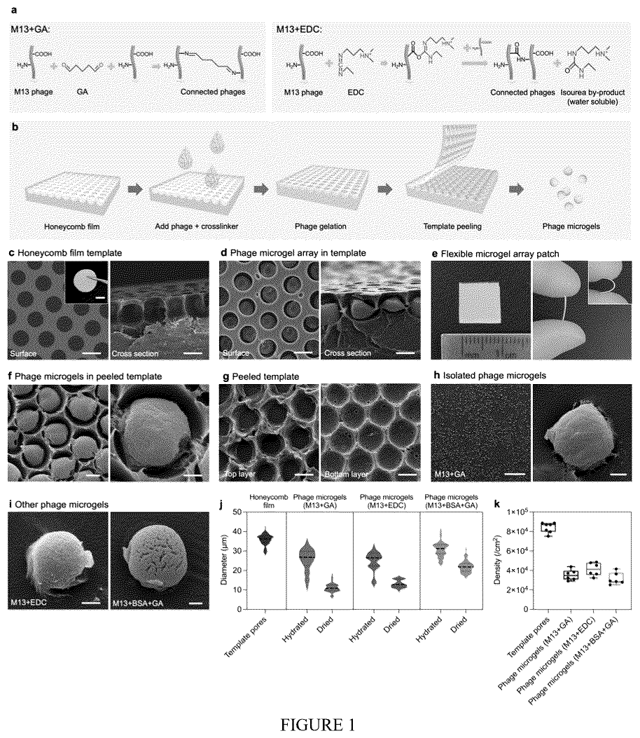

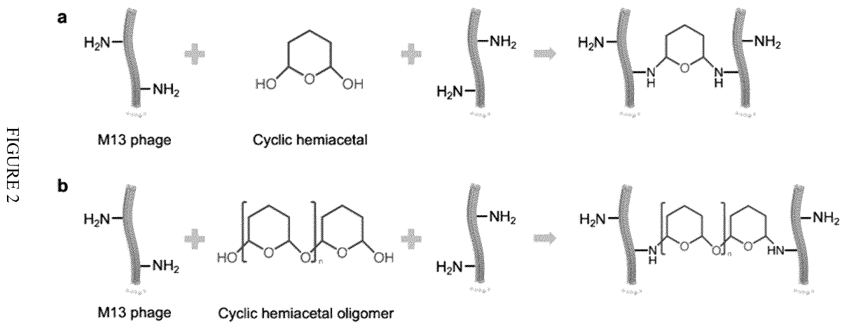

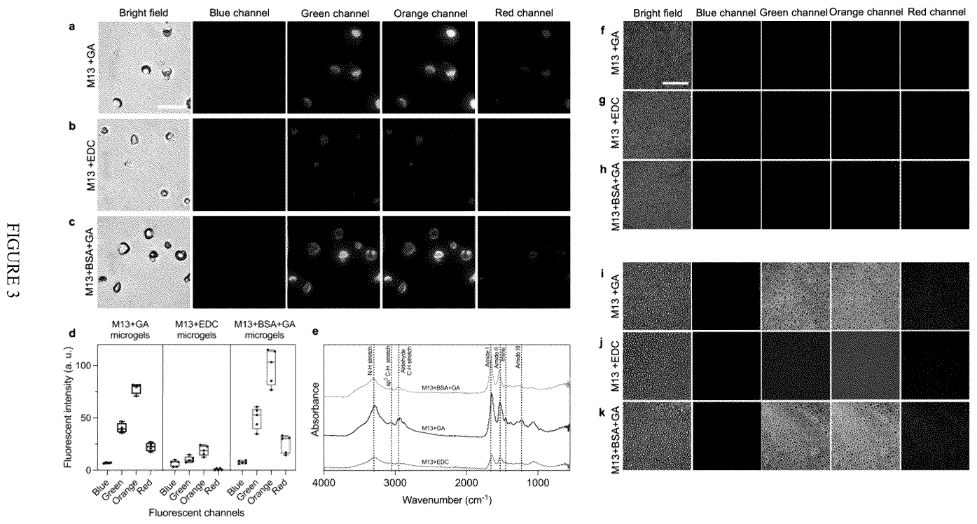

Generation of bacteriophage microgels. The gelation of phage aqueous suspension is based on the crosslinking reaction between M13 filamentous phage and a small molecule chemical crosslinker. The crosslinker, glutaraldehyde (GA), can react with multiple functional groups on the phage coat protein, notably amino groups on the lysine residues^{15,27,28}. As shown in FIG. 1a, reaction 1, a GA molecule reacts with 2 amino groups on two phage capsids and forms Schiff bases connecting these two phages. It is worth mentioning that GA in aqueous solution is not limited to regular monomeric formation. For example, cyclic hemiacetal and cyclic hemiacetal oligomer are common forms of GA which can react with amine as well and form ether groups (FIG. 2). All these possible reactions proceed simultaneously leading to a crosslinked network of phage nanofilaments. The formation of these side products, in addition to the well-documented self-polymerization of GA and the strong autofluorescence of the final gel¹⁵, make GA a non-ideal choice for certain applications. Therefore, a second small molecule crosslinker 1-Ethyl-3-(3-dimethylaminopropyl) carbodiimide (EDC) was explored, which proved capable of crosslinking phage through a different crosslinking mechanism²⁹. An EDC molecule first reacts with a carboxyl group on the phage coat protein and forms an amine-reactive intermediate that quickly reacts with an amine group on another phage coat protein to form an amide bond between two phages (FIG. 1a, reaction 2)^{30,31}. It is noteworthy that EDC molecule were not incorporated into the final crosslinked product. Instead, EDC took oxygen atoms from carboxyl groups on phages and formed water-soluble isourea by-product which can be easily washed out. This intermediate role of EDC is the fundament of fabricating phage-exclusive microgels. This mechanism was found to be effective at controlling the optical properties of the microgels, as will be discussed in FIG. 3, which potentially brings different applications to phage microgels.

A single M13 phage exhibits abundant amine and carboxyl groups (8,100 and 10,800, respectively) on its protein coat³², providing rich reaction sites for crosslinking reactions. The crosslinked phage virions form a network, resulting in the gelation of the phage aqueous suspension. It was observed that M13 and EDC mixture needs less than 12 hrs to gel while the same concentration of GA takes about 24 hrs to gel.

The number of reactive subset of amine and carboxylic groups from on the M13 coat proteins were calculated based on the amino acid sequence summarized from reference³². M13 capsid is composed of approximately 2700 copies of pVIII protein, and there are reactive amine groups from 2 lysine and 1 alanine (N-terminal) on each pVIII protein³². Therefore, there should be approximately 8,100 reactive amine groups (2,700×3). The reactive carboxyl groups are provided from 2 aspartic acids and 2 glutamic acids in each pVIII protein³². Therefore, there are approximately 10,800 reactive carboxyl groups (2,700×4).

It is noteworthy that heat and organic solvents are commonly involved during microparticle preparation or isolation^{23,33}, which have irreversible detriments for biomaterials. Therefore, manufacturing viral microgels without losing bioactivity would have been exceptionally challenging without developing a suitable microgel manufacturing method. Herein, proposed is a biomaterial-friendly approach for the parathion of pure and hybrid phage microgel. The phage microgels were gelled in and isolated from a polystyrene honeycomb film containing uniform open-ended spherical micropores throughout the film surface, as illustrated in FIG. 1b.

The honeycomb films here were prepared via a well-established approach known as the breath figure method^{34,35}. This is an easy-approachable and rapid method to fabricate the large-scale template with single-layer, closely-packed, and homogenous micropores without any large equipment. The size of the template can be changed by applying different volumes of polystyrene solution to the glass slide. In the current experiments, 600 μL of polystyrene solution can generate a honeycomb film with a diameter of 2.5 cm in 20 mins. The micropores were uniformly rounded and the cross-sectional Scanning Electron Microscope (SEM) image in FIG. 1c indicates that the inner pores exhibited open-ended spherical shape. The inner diameter of the template pores was 35.73±2.86 μm, as measured by electron micrographs.

To fabricate phage microgels, a mixture of M13 suspension and crosslinker (GA/EDC) was cast on a plasma-treated polystyrene honeycomb film where the mixture fills inside the micropores (FIG. 1b). After removing the redundant phage solution on the template surface by a glass slide, the film was transferred into a sealed humid container at 4° C. for 1 day for gelation. As shown in FIG. 1d, the original phage suspension inside the pores successfully turned into an ordered array of solid phage microgels. The honeycomb template is a thin round polystyrene film, which means the composite film loading the phage microgel array is flexible. FIG. 1e shows a composite film (phage microgels inside the template film) tailored into a 1 cm² square which can easily bend. These properties make the film an excellent patch integrating phage microgels for further antimicrobial and biosensing studies.

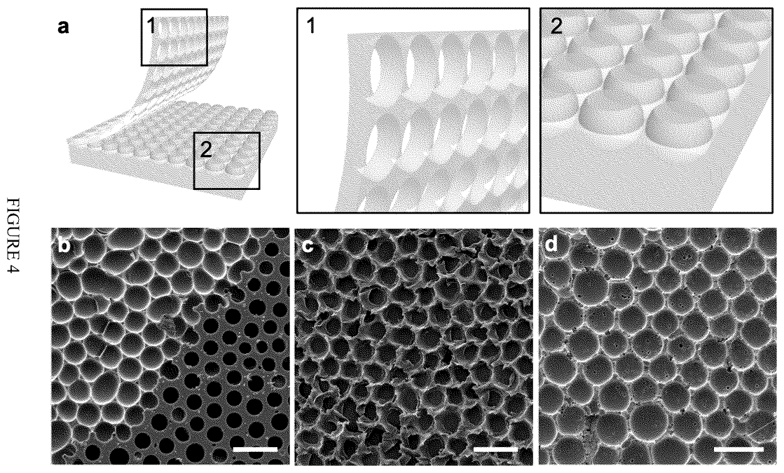

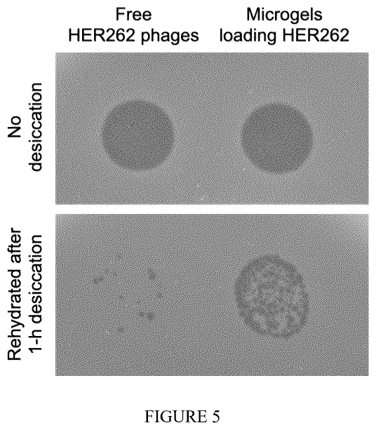

Moreover, the microgel array inside the template is detachable. A piece of adhesive tape was used to stick on the composite honeycomb film surface to then peel off the top half of the pores (FIG. 4a). Consequently, the top half of the pores was attached on the tape and the microgels inside the film were exposed on the bottom film layer without damage (FIG. 1f). Phage microgels were conveniently isolated from the template by immersing the film in sterile water and sonicating. FIG. 1g shows the permeable polystyrene pore network on the tape and the shallow pore structure left on the honeycomb film after peeling and sonication. The microgels were detached successfully and transferred to water phase. FIG. 4b reveals the edge of the pealing area where the top left of the image is the peeled area of the honeycomb film showing shallow pores and the bottom right area is the unpeeled deep pores. The peeling procedure effectively separated the honeycomb film (FIG. 4c-d). Phage microgels were suspended in Millipore water. FIG. 1h-i shows the isolated phage acrogel microbeads which are the M13+GA/EDC microgels after critical point drying, proving the microgel array inside the template are detachable. In addition to pure phage microgels, bovine serum albumin (BSA) was also added to M13+GA solution to demonstrate application of the developed method to fabricate phage-protein hybrid phage microgels, which can further expand the functionality. BSA efficiently provided abundant amino groups and carboxyl groups to consume excessive crosslinker molecules and preserve the bioactivity of M13 phages, which will be illustrated in FIG. 5. In addition, the gelation reaction is accelerated from over 12 hrs to about 30 mins. Although template methods have been used for making microparticles in the past with calcination³⁵, the manufacturing method disclosed was designed to enable production of microscale colloidal soft matter, namely phage microgels, in the form of a peelable patch or a suspension. This method is high-throughput, heat-free, and solvent-free, which makes it especially advantageous to keep biomolecules active. The size and shape of the microparticles is determined to the template pore shape and size. Fortunately, there are already abundant studies extending the breath figure method to fabricate honeycomb films containing ordered pores at different sizes and shapes³⁶⁻³⁸.

Size distribution, porosity and preparation efficiency of phage microgels. FIG. 1j presents the size distribution of the template pores and phage microgels. The M13 phage microgels crosslinked by GA and EDC separately show a similar size distribution, which is smaller than the template pores (25.34±5.72 μm and 24.39±4.92 μm respectively). It is noteworthy that the size range of the microgels has a broader distribution compared to the template pores. This might be caused by shrinkage during gelation and possible breakage in the isolation process. Based on the phage concentration used (5×10¹³ PFU/mL) and the average microgel size, it can be estimated that each phage microgel is composed of over 7×10⁵ M13 phages. The average size of phage microgels containing BSA is larger (30.77±3.83 μm) and the size range is narrower. The addition of BSA likely makes the microgels denser so the shrinkage and damage during the whole process is reduced, which is reflected in the average size and size distribution.

The porosity of these phage microgels was evaluated by measuring the size change between hydrated and air-dried states. The GA and EDC microgels decreased in diameter to 11.13±2.32 μm and 13.16±1.986 μm after dehydration, showing 91.5% and 84.3% volume reduction, respectively. The high-volume reduction of phage microgels suggests high porosity. The microgels with added BSA had significantly less shrinkage, maintaining an average size of 21.97±3.04 μm (63.60% volume size reduction), indicating denser, less porous microgels.

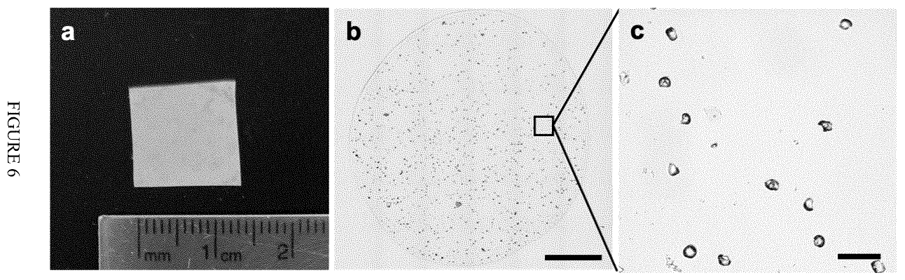

The preparation efficiency of the phage microgels was investigated by calculating the microgel count obtained from every square centimeter of the template (details in FIG. 6). Firstly, the size of honeycomb film Sₜₑₘₚₗₐₜₑ was measured before microgel isolation (FIG. 6a). After collecting all the microgels into 1 mL of water from the peeled template, 5 μL of microgel suspension was dropped on the glass slide and snapped high-resolution images (FIG. 6b-c). The amount of microgels in that droplet (N_{microgel}) was counted, and the microgel preparation efficiency was calculated: η=N_{microgel}×200/Sₜₑₘₚₗₐₜₑ.

The honeycomb film template contained 83862+5241 micropores/cm². The usage of GA crosslinker produced 35295±5490 phage microgels/cm² while EDC crosslinker produced 41226±6878 microgels/cm² (FIG. 1k). The addition of BSA produced similar results (31431±6185 micropores/cm²). The number of microgels produced from the template is lower than the template pore density (42.1%, 49.2% and 37.5%). This could be a result of partial filling of phage suspension into the pores or the loss during isolation process. In summary, over 3.5×10⁴ phage microgels was obtained from every square centimeter of this template where each microgel contains more than 3.8×10⁵ phage particles, constituting a phage community of 10¹⁰ in total. Every film made was over 5 cm², allowing for the production of 175,000 phage microgels in a single day. In addition, more than 10 films can be applied to produce microgels simultaneously, demonstrating the high-throughput ability of this method.

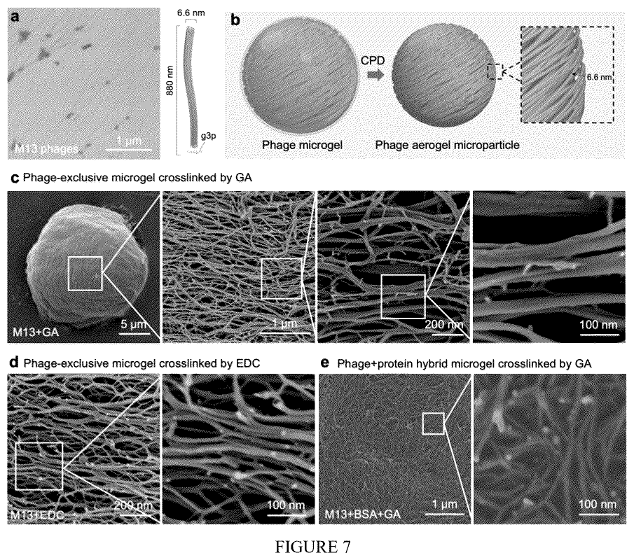

Highly aligned nanofibrous texture of phage microgels. As shown in FIG. 7a, M13 phages are high aspect ratio nanofilaments (length=880 nm, width=6.6 nm) with a relatively large tip, where five copies of the bacterium-binding protein (g3p) protrude from one end. The size of the small molecule crosslinker GA (M_{w}=100.11 g/mol) and EDC (M_{w}=191.70 g/mol) is negligible in comparison. It is hypothesized that the phage microgels have nanofibrous texture which are crosslinked filamentous phages, as shown in the schematic image in FIG. 7b.

As shown in the SEM images (FIG. 7c), the M13 aerogel microparticles crosslinked by GA showed a sophisticated thread pattern. At higher magnification, nanofibers were observed aligning at a single orientation overall where these nanofibers (width between 7 nm and 20 nm) fit the width of a single M13 phage. The uneven width of fibers could likely be a result of the inevitable 3-nm Pt coating for SEM or the occasional lateral binding of multiple phages. The M13 acrogel microparticles crosslinked by EDC showed a similar ordered nanostructure (FIG. 7d). The bright dots in these images are the g3p bacterium-binding sites; these are large protruding proteins that stand out in electron micrographs. In summary, FE-SEM imaging showed densely packed, self-assembled nanofilaments that formed highly-ordered nanofibrous network during the gelation process.

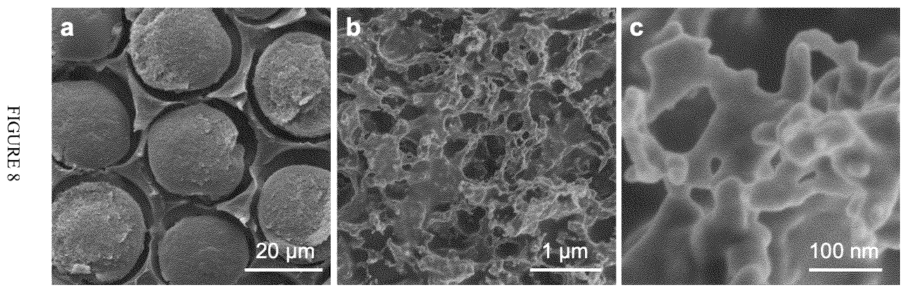

It is noteworthy that the phage alignment in the hybrid phage-protein (M13+BSA) microgels crosslinked by GA was distinct. The phage nanofilaments in the hybrid microgels were partially embedded in BSA with no particular order (FIG. 7e). Adding BSA to the microgel system accelerated the gelation process from 12 hrs to 30 mins, leaving insufficient time for phages to self-assemble. The g3p proteins are still exposed on the microgel surface, providing bacterium-binding sites. Moreover, pure BSA protein microgels crosslinked by GA were investigated to compare the nanostructure with that of phage microgels and confirmed that the observed order in the M13 microgels was caused by the M13 nanofilaments and not by the crosslinker or the dehydration procedures. The BSA microgels were processed by the same preparation and dehydration procedures and showed no sign fibrous nanostructures. The SEM images of these microgels (FIG. 8) shows irregularly rough surfaces, unlike the nanofibrous texture of the phage microgels.

In conclusion, the phage-exclusive microgels exhibit high porosity, potentiating their strong loading capacity of proteins, phages, and small molecules. The homogenous nanofibrous texture along the same orientation is the direct evidence that the microgels are composed by phages solely crosslinked by small molecule. The addition of protein interfered the order alignment of phages, but played an important role in preserving the phage bioactivity which will be illustrated later.

Autofluorescence of phage microgels can be tuned by using different crosslinkers. As shown in FIG. 3a, the phage microgels made with GA showed significant autofluorescence in four channels. This phenomenon is associated with electronic transitions such as the n−π* transitions of C=N in the Schiff's base generated from crosslinking reactions^{15,39}. This autofluorescence potentiates non-destructive imaging capability of the microgels. However, fluorescence of phage microgels can be troublesome in some application scenarios, for example certain biosensing applications that rely on fluorescence to detect the target analytes. For this reason, microgels of phage crosslinked with an alternative small molecule crosslinker were explored, namely EDC, that do not exhibit autofluorescence (FIG. 3b). The amide bonds formed between M13 phage and EDC cause a much weaker fluorescent signal, thus expanding the range of applications for the microgels. In the green and orange channels, the fluorescence of M13+GA and M13+BSA+GA microgels was 294.7% and 320.9% higher than M13+EDC microgels. The largest difference appeared in the red channel where GA-crosslinking microgels had fluorescent signal but the fluorescence of EDC-crosslinking microgels was not observed. Both GA- and EDC-crosslinking phage microgels showed low-fluorescence in the blue channel, and the strongest fluorescence was observed in the orange channel (94.7% and 82.5% higher than their second strongest channel, green).

For scenarios where a strong fluorescence signal is anticipated to be advantageous, BSA can be added to M13+GA microgels to participate in gelation to enhance the fluorescent signal (FIG. 3c). The quantified fluorescent intensity of those microgels is illustrated in FIG. 3d. The addition of BSA followed the same trend at different channels as pure phage microgels, and enhanced the fluorescent intensity compared to the GA microgels (23.5% higher at green channel and 26.1% higher at orange channel). Fourier transform infrared (FTIR) spectra further confirmed the functional groups on different phage microgels (FIG. 3e). The spectra of M13+GA, M13+EDC and M13+BSA+GA microgels are very similar because of the abundant functional groups on the proteinous capsid of phages. All three spectra showed typical peaks at 1660, 1530 and 1230 cm⁻¹, representative for the amide I, II and III bonds on protein capsids. The three unique peaks showing in GA-crosslinking microgels rather than EDC-crosslinking microgels are at around 3050, 2950 and 1450 cm⁻¹ corresponding to sp² C-H stretch, aldehyde C-H stretch, and imine (C═N) bonds respectively.

In addition, the gelation procedure was monitored using fluorescence microscopy to confirm that the fluorescence signal is the result of gelation and not inherited from the phage building blocks, templates, or crosslinkers. During the gelation process, a distinct change in fluorescence was observed using microscopy with four different optical filter sets. As shown in FIG. 3f-h, 5×10¹³ PFU/mL of M13 phage suspension with 0.1 M of either crosslinker inside the honeycomb film showed no fluorescence. After the gelation of phage with GA was complete (with or without the addition of BSA), the composite films emitted obvious fluorescent signal at green, orange, and red channels. The signal in blue channel was very weak. This change is also observed in the composite film with EDC where its fluorescence signal at these four channels is significantly lower (FIG. 3i-k). This observation confirms that the crosslinking reactions are the sole reason for autofluorescence and suggests that using different crosslinkers can aid in fine-tuning the fluorescence signal to match the requirements of different application scenarios.

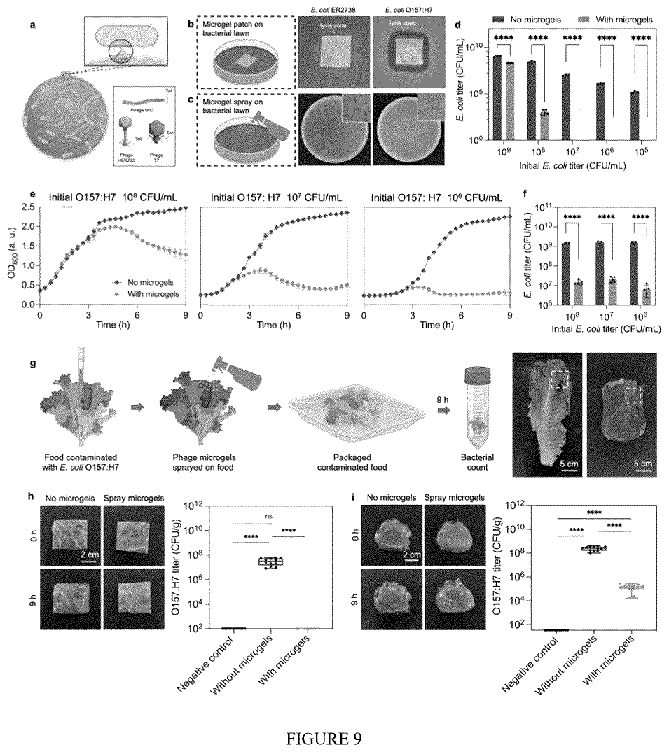

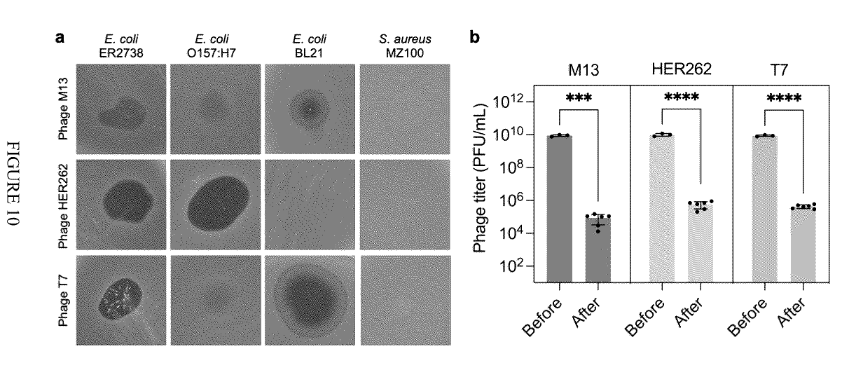

Targeted antimicrobial functions of phage microgel patches and sprays. It was hypothesized that the phage microgels inherited the antimicrobial activity of their phage building blocks and are able to specifically target host bacteria. To investigate the antimicrobial performance of pure and potentially hybrid phage microgels, M13 was used along with two virulent E. coli phages, namely T7 and HER262, which have strong and specific killing action but different geometric shapes and mechanisms of infection (details in FIG. 9a and FIG. 10a). Further, it was demonstrated that the hydrated environment in microgels can protect desiccation-sensitive phages. As shown in FIG. 10b, the titers of phages M13, HER262 and T7 all decreased about 4 logs after drying for 1 hr at room temperature and rehydrating.

Test of specific targeting ability and desiccation sensitivity of phages. For example, phages M13 (Inoviridae, filamentous), HER262 (Myoviridae, long tailed), and T7 (Podoviridae, short tailed), are all E. coli phages and they cannot infect other bacterial species such as Staphylococcus aureus (FIG. 10). From the three E. coli strains evaluated, M13 phage formed mild lysis zones on the lawns of E. coli ER2738 and E. coli BL21, while phage HER262 showed lysis zones on strains ER2738 and O157:H7. Phage T7 was able to significantly lyse E. coli ER2738 and E. coli BL21. It is noteworthy that the highly specific bactericidal action of phages means that to control the population of multiple species, a mixture of phages has to be used⁴⁸. However, the advantage of this specific killing action is preserving the beneficial bacteria in food that are responsible for maintaining the taste and texture of many food products^{49,50}.

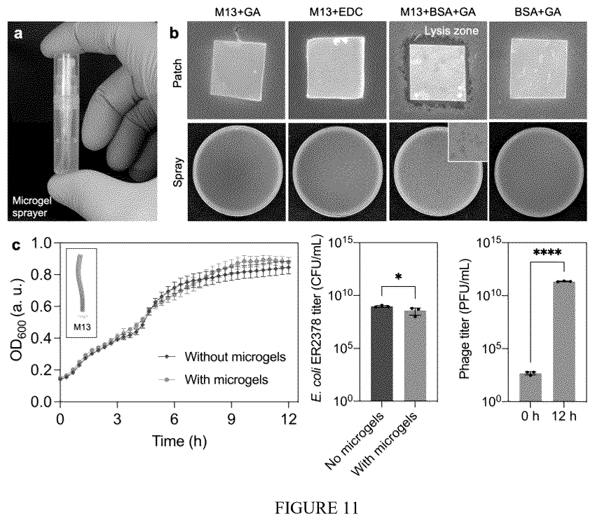

The antimicrobial activity of phage microgels was demonstrated in three biocontrol scenarios: an undetached microgel array in the template as an antimicrobial patch, a microgel sprayer (FIG. 11a), and the addition of microgels directly to a bacterial-contaminated liquid. The microgel spray directly used the microgels suspension, containing over 3×10⁴ microgels/mL and all microgels were washed twice. Free phage was not detectable in the eluent after this point. Initially, the infectivity of pure M13 phage microgels was evaluated. It was found out that these two types of microgels did not show obvious infectivity to E. coli ER2738 (M13 phage's natural host), neither as a patch or spray (FIG. 11b). The lack of obvious bioactivity stems mainly from the fact that M13 has a low antimicrobial activity even in free suspension form¹⁰. Intramolecular crosslinking can further decrease this already low activity. On the contrary, M13+BSA+GA hybrid microgels maintained their infectivity and the corresponding patch formed lysis zone around the edges on a lawn of E. coli ER2738 and sprayed microgels formed plaques on the bacteria lawn (FIG. 11b), clearly indicating antimicrobial activity. The non-infectivity of BSA+GA microgels confirmed that the bioactivity of M13+BSA+GA microgels is not bound to BSA or reacted crosslinker (FIG. 11b). Therefore, it is possible that the abundant amino groups and carboxyl groups offered by BSA consumed excessive crosslinker molecules, minimizing the intramolecular crosslinking within phages and eventually protecting the bioactivity of phages. FE-SEM imaging showed the bacterial-binding sites, g3p, were displayed on the surface of phage microgels, so these microgels were expected to bind multiple E. coli ER2738 as shown in the schematic image FIG. 9a. Even though M13+BSA microgels successfully retained the bioactivity, the hybrid phage microgels cannot kill E. coli sufficiently considering that M13 is a weakly antimicrobial phage to begin with⁴⁰. FIG. 11c shows that adding M13+BSA microgels to a ER2738-contaminated nutrient environment cannot prevent the growth of E. coli, again indicating that a stronger virulent phage should be used for bactericidal activity. It is noteworthy that M13 is an attractive phage for use as structural component (mainly due to its shape and readily available toolkits for genetic engineering) but is not commonly the phage of choice for biocontrol of bacterial contamination/infections where bactericidal activity is desired.

Integrating phages as microgels was expected to offer four main advantages compared to applying a phage suspension for biocontrol. One is desiccation control (FIG. 5). Another is that microgels can achieve very high local concentration. It was calculated that based on the titer of phage suspension needed to fabricate microgels (5×10¹³ PFU/mL), each microgel contains more than 3.8×10⁵ of M13 phages. This not only benefits the antimicrobial application, but also provides massive recognition sites when using modified phages to construct microgel biosensors. Additionally, the nanofibrous structure of M13 phage microgels provides strong loading capacity to load other antimicrobial factors, such as antibodies, small molecule inhibitors, or other bacteriophages. Finally, microgels offer a higher surface area compared to a bulk microgel, thus increasing the contact area between phage and its bacterial host which is expected to increase the antibacterial potency of the microgel over the same weight of bulk hydrogel.

Hybrid phage microgels targeting multidrug-resistant bacteria. The microgels were embedded with strong virulent phages to enhance the bactericidal ability of the microgels. Virulent phages, a class of phage with strong antimicrobial action, are different in physical structure and mechanism of antibacterial action than filamentous phage. Preserving the antibacterial action of virulent phages inside the gels, however, is a major challenge because their host recognition/binding sites are often located asymmetrically on tip of their tail fibers (such as phage HER262 and T7 which were previously shown in FIG. 9a). The relatively fragile tail fibers can be easily damaged through processing, or blocking. This challenge was addressed by not only optimizing the chemistry, but also by working at the microscale, thus increasing the surface area for phage action.

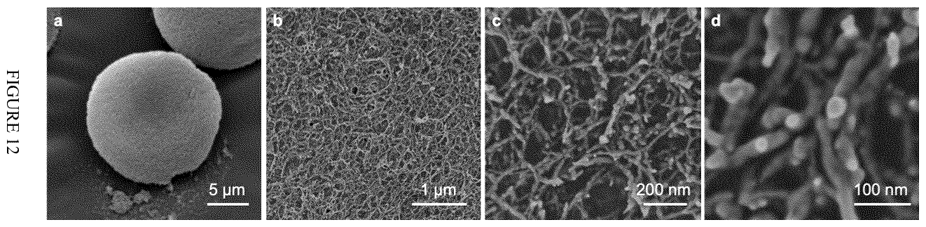

To minimize the intramolecular crosslinking within phages, the concentration of GA was decreased from 0.1 M to 0.02 M. At this low concentration of crosslinker, the phage suspension cannot gel without the presence of BSA. The first virulent phage added to the M13+BSA microgels was phage HER262 (1×10¹⁰ PFU/mL) that targets multidrug-resistant E. coli O157:H7, a common bacterial contaminant on meats and lettuces^{41,42}. The SEM images confirmed the formation of hybrid microgels (FIG. 12). Comparing to the capsulated nanofibrous structure of M13+BSA+GA microgels (FIG. 7e), the surface nanostructure of M13+HER262+BSA+GA microgels displayed nanodots matching the capsid size of phage HER262 (˜50 nm), supporting retention of HER262 antimicrobial activity through its ability to target E. coli O157:H7. As shown in FIG. 9b, the unseparated hybrid phage HER262 microgels in the patch formed lysis zones on the lawns of both E. coli ER2738 and E. coli O157:H7. The microgel formed clear plaques, indicative of antimicrobial activity, when sprayed on both lawns (FIG. 9c).

To evaluate the bacteria-killing ability of the microgels in liquid, E. coli O157:H7 and phage microgels were incubated together in two environments: phosphate-buffered saline (PBS) simulating a nutrient-deficient environment, and nutrient tryptic soy broth (TSB) simulating a nutrient-rich environment.

For the nutrient-deficient environment, PBS was used to dilute the pre-culture into 10⁸, 10⁷, 10⁶, and 10⁵ CFU/mL. Phage microgels were then added to the diluted bacterial suspensions at a final concentration of ˜1500 microgels/mL. As shown in FIG. 9d, E. coli O157:H7 maintained the same concentration level in PBS without microgels after 9 hrs. On the contrary, when microgels were added, they killed all bacteria within 9 hrs when the initial concentration of E. coli O157:H7 was below 10⁷ CFU/mL, and decreased the concentration 6 logs when initial concentration was high, 10⁸ CFU/mL.

In nutrient TSB, phage microgels also showed the ability to prevent bacterial growth, but at a much faster rate. This is expected because the phage antimicrobial activity is closely tied to the physiological state of the host bacteria. TSB was used to dilute the pre-culture into 10⁸, 10⁷, 10⁶ CFU/mL and monitored the optical density at a wavelength of 600 nm (OD₆₀₀) in the suspension to evaluate bacterial growth. As shown in FIG. 9e, the phage microgels restrained the increase of OD₆₀₀ in 4 hrs regardless at high and low contamination loads. The bacterial titers were further quantified after 9 hrs (FIG. 9f). Phage microgels prevented the growth of E. coli O157:H7, maintaining the bacterial titer between 10⁶ and 10⁷ CFU/mL while all the controls reached 10⁹ CFU/mL.

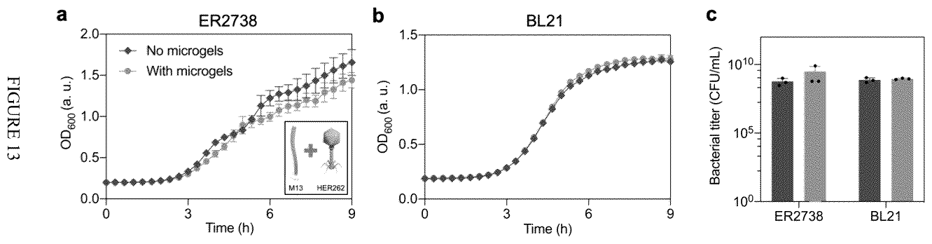

In summary, phage microgels displayed excellent antimicrobial ability regardless of nutrient in the environment, especially in the nutrient-deficient environment where bacterial propagation was inhibited. Moreover, to demonstrate specific bactericidal activity of the microgels, phage microgels were incubated with ER2738 and BL21 at the initial titer of 10⁶ CFU/mL and the bacterial solutions showed same strong growing trend regardless of the participation of microgels (FIG. 13), illustrating the specific targeting of these microgels.

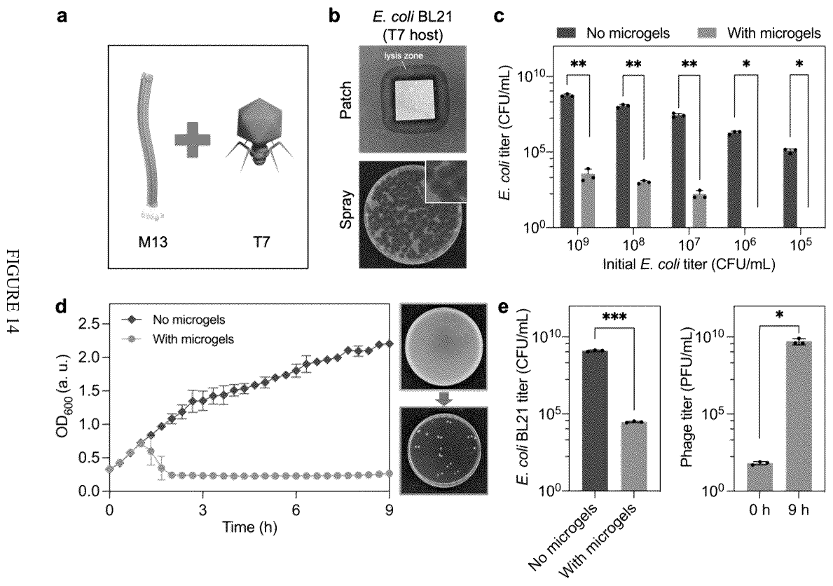

It was confirmed that the antimicrobial activity was independent of the phage used as long as a virulent phage was used, by demonstrating the results with the virulent phage T7 (FIG. 14a). The patch and spray made with T7-embedded phage microgels showed lysis zones on the lawn of BL21 (FIG. 14b). The microgels can decrease the titer of BL21 by at least 6 logs (FIG. 14c). In nutrient TSB solution, T7-embedded phage microgels can still prevent the bacterial growth and caused a 5 log difference (FIG. 14d-e), which proved the strong function of phage microgels as delivery vehicle.

Phage microgel spray for food product safety. After verifying the antimicrobial activity of HER262-embedded phage microgels against E. coli O157:H7, these microgels were used to inhibit bacterial contamination in two completely different food matrices. As illustrated in FIG. 9g, the lettuce was first contaminated with E. coli O157:H7 at 10⁶ CFU/g, followed by spraying with phage microgels. The lettuce was then covered with food wrap and placed in room temperature for 9 hrs. The second day, the lettuce was immersed in 10 mL of PBS and vortexed for 2 mins to collect the live bacteria. As shown in FIG. 9h, it is difficult to visually differentiate between the lettuce leaves with different treatment, but the bacterial concentration in the collected solution is significantly different. The contaminated lettuce with no microgel treatment reached an average contaminant load of 3.3×10⁷ CFU/g after 9 hrs. For the contaminated lettuce sprayed with microgels, the bacterial titer dropped to undetectable level (<100 CFU/g, up to 6 log reduction). The same antimicrobial phenomenon was observed when testing artificially contaminated meat samples with a similar treatment. The meat samples showed no visual difference. The O157:H7-contaminated meat sprayed with water reached 2.5×10⁸ CFU/g, For the contaminated meat sprayed with microgels, the bacterial titer dropped to 1.4×10⁵ CFU/g, indicating that the microgels killed 99.94% of the drug-resistant bacteria.

Conclusion. The main discoveries and contributions of this work are (1) establishment of virus-built microparticles, (2) development of a biomolecular-friendly high-throughput preparation method for diverse phage microgels, (3) highly-aligned nanofibrous texture of phage-exclusive microgels, (4) tunable autofluorescence, and (5) the application of the phage-protein hybrid microgel patch and microgel sprays for biocontrol. The high-throughput method proposed here combined with honeycomb template casting with peel isolation. It produced over 35,000 phage microgels in every square centimeter template with each microgel containing half a million phages. This method can be extended to prepare most types of microgels efficiently, but it is particularly suited to heat/solvent-sensitive microgels as it is simple, heat-free, and solvent-free, which is especially useful to keep biomolecules and proteinaceous materials functional. The nanofilamentous building blocks self-assembled forming a highly aligned nanofibrous structure where single phage filaments could be observed using an electron microscope. Addition of BSA protein in microgels added additional flexibility in design, namely, to tune the fluorescence and preserve phage bioactivity. Furthermore, strong virulent phages were combined into the microgels and the resulting microgel patch, microgel spray and microgel suspension were proven highly effective in their antimicrobial action. Specifically, the contaminant load of the multidrug resistant Escherichia coli O157:H7 in food products were reduced by 6 logs after spraying phage microgels. It is further demonstrated that aside from packing a high density of antimicrobial virions, the microgels also protected against desiccation. Every year, it is estimated that 600 million people fall ill due to the consumption of contaminated food. This attributes to 420,000 annual deaths globally and E. coli contamination is considered a major factor⁴³. Incorporating the antimicrobial microgels or patches into packaging, sprays in grocery store produce sections, and in household decontaminating products can effectively inhibit bacterial contamination in a human-friendly manner that will ultimately reduce foodborne illnesses, deaths and associated economic loss.

While the present disclosure has been described with reference to examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present disclosure is found to be defined differently in a document incorporated herein by reference, the definition provided herein is to serve as the definition for the term.

### CITATIONS

1. Salmond, G. P. C. & Fineran, P. C. A century of the phage: Past, present and future. Nat. Rev. Microbiol. 13, 777-786 (2015).
2. Meng, R. et al. Structural basis for the adsorption of a single-stranded RNA bacteriophage. Nat. Commun. 10, (2019).
3. Hsu, B. B. et al. Dynamic Modulation of the Gut Microbiota and Metabolome by Bacteriophages in a Mouse Model. Cell Host Microbe 25, 803-814.e5 (2019).
4. World Health Organization. WHO's first ever global estimates of foodborne diseases find children under most one third of deaths. (2015). Available at: https://www.who.int/news/item/03-12-2015-who-s-first-ever-global-estimates-of-foodborne-diseases-find-children-under-5-account-for-almost-one-third-of-deaths. (Accessed: 7th February 2022)
5. Food and Drug Administration. GRAS notice 755, Preparation containing two bacterial phages specific to Escherichia coli O157. (2018). Available at: https://www.fda.gov/media/117249/download. (Accessed: 24th August 2021)
6. Food and Drug Administration. GRAS Notice 834, Preparation containing bacterial phages specific to shiga-toxin producing Escherichia coli. (2019). Available at: https://www.fda.gov/media/133519/download. (Accessed: 24th August 2021)
7. Lucera, A., Costa, C., Conte, A. & Del Nobile, M. A. Food applications of natural antimicrobial compounds. Front. Microbiol. 3, 1-13 (2012).
8. Wang, X. et al. Phage combination therapies for bacterial wilt disease in tomato. Nat. Biotechnol. 37, 1513-1520 (2019).
9. Moye, Z. D., Woolston, J. & Sulakvelidze, A. Bacteriophage applications for food production and processing. Viruses 10, 1-22 (2018).
10. Bayat, F., Didar, T. F. & Hosseinidoust, Z. Emerging investigator series: bacteriophages as nano engineering tools for quality monitoring and pathogen detection in water and wastewater. Environ. Sci. Nano 8, 367-389 (2021).
11. Smith, G. P. Filamentous Fusion Phage: Novel Expression Vectors that Display Cloned Antigens on the Virion Surface. Science 228, 1315-1317 (1985).
12. Souza, G. R. et al. Three-dimensional tissue culture based on magnetic cell levitation. Nat. Nanotechnol. 5, 291-296 (2010).
13. Chung, W. J. et al. Biomimetic self-templating supramolecular structures. Nature 478, 364-368 (2011).
14. Oh, J. W. et al. Biomimetic virus-based colourimetric sensors. Nat. Commun. 5, 1-8 (2014).
15. Peivandi, A., Tian, L., Mahabir, R. & Hosseinidoust, Z. Hierarchically Structured, Self-Healing, Fluorescent, Bioactive Hydrogels with Self-Organizing Bundles of Phage Nanofilaments. Chem. Mater. 31, 5442-5449 (2019).
16. Peivandi, A. et al. Inducing Microscale Structural Order in Phage Nanofilament Hydrogels with Globular Proteins. ACS Biomater. Sci. Eng. In press (2021).
17. Jackson, K., Peivandi, A., Fogal, M., Tian, L. & Hosseinidoust, Z. Filamentous Phages as Building Blocks for Bioactive Hydrogels. ACS Appl. Bio Mater. 4, 2262-2273 (2021).
18. Guan, Y. & Zhang, Y. PNIPAM microgels for biomedical applications: From dispersed particles to 3D assemblies. Soft Matter 7, 6375-6384 (2011).
19. Rossow, T. et al. Controlled synthesis of cell-laden microgels by radical-free gelation in droplet microfluidics. J. Am. Chem. Soc. 134, 4983-4989 (2012).
20. Seiffert, S. & Weitz, D. A. Microfluidic fabrication of smart microgels from macromolecular precursors. Polymer (Guildf). 51, 5883-5889 (2010).
21. Yun, Y. H., Goetz, D. J., Yellen, P. & Chen, W. Hyaluronan microspheres for sustained gene delivery and site-specific targeting. Biomaterials 25, 147-157 (2004).
22. Mao, A. S. et al. Deterministic encapsulation of single cells in thin tunable microgels for niche modelling and therapeutic delivery. Nat. Mater. 16, 236-243 (2017).
23. Headen, D. M., Aubry, G., Lu, H. & García, A. J. Microfluidic-based generation of size-controlled, biofunctionalized synthetic polymer microgels for cell encapsulation. Adv. Mater. 26, 3003-3008 (2014).
24. Li, F., Lyu, D., Liu, S. & Guo, W. DNA Hydrogels and Microgels for Biosensing and Biomedical Applications. Adv. Mater. 32, 1-9 (2020).
25. Leung, V., Groves, L., Szewczyk, A., Hosseinidoust, Z. & Filipe, C. D. M. Long-Term Antimicrobial Activity of Phage-Sugar Glasses is Closely Tied to the Processing Conditions. ACS Omega 3, 18295-18303 (2018).
26. Hsu, B. B. et al. In situ reprogramming of gut bacteria by oral delivery. Nat. Commun. 11, 1-11 (2020).
27. Chen, P. Y. et al. Versatile three-dimensional virus-based template for dye-sensitized solar cells with improved electron transport and light harvesting. ACS Nano 7, 6563-6574 (2013).
28. Ohmura, J. F. et al. Highly adjustable 3D nano-architectures and chemistries: Via assembled 1D biological templates. Nanoscale 11, 1091-1101 (2019).
29. Courchesne, N. M. D. et al. Assembly of a bacteriophage-based template for the organization of materials into nanoporous networks. Adv. Mater. 26, 3398-3404 (2014).
30. Grabarek, Z. & Gergely, J. Zero-length crosslinking procedure with the use of active esters. Anal. Biochem. 185, 131-135 (1990).
31. Timkovich, R. Detection of the stable addition of carbodiimide to proteins. Anal. Biochem. 79, 135-143 (1977).
32. Chung, W. J., Lee, D. Y. & Yoo, S. Y. Chemical modulation of M13 bacteriophage and its functional opportunities for nanomedicine. Int. J. Nanomedicine 9, 5825-5836 (2014).
33. Li, X. et al. A Bottom-Up Approach To Fabricate Patterned Surfaces with Asymmetrical TiO2 Microparticles Trapped in the Holes of Honeycomblike Polymer Film. J. Am. Chem. Soc. 113, 3736-3739 (2011).
34. Widawski, G. & Rawiso, M. Self-organized honeycomb morphology of star-polymer polystyrene films. Nature 369, 387-389 (1994).
35. Zhu, C., Tian, L., Liao, J., Zhang, X. & Gu, Z. Fabrication of Bioinspired Hierarchical Functional Structures by Using Honeycomb Films as Templates. Adv. Funct. Mater. 28, 1-8 (2018).
36. Daly, R., Sader, J. E. & Boland, J. J. Taming Self-Organization Dynamics to Dramatically Control Porous Architectures. ACS Nano 10, 3087-3092 (2016).
37. Takehiro Nishikawa et al. Fabrication of Honeycomb Film of an Amphiphilic Copolymer at the Air-Water Interface. Langmuir 18, 5734-5740 (2002).
38. Wang, W. et al. Deterministic Reshaping of Breath Figure Arrays by Directional Photomanipulation. ACS Appl. Mater. Interfaces 9, 4223-4230 (2017).
39. Wei, W. et al. Preparation and application of novel microspheres possessing autofluorescent properties. Adv. Funct. Mater. 17, 3153-3158 (2007).
40. Waldor, M. K. & Mekalanos, J. J. Lysogenic conversion by a filamentous phage encoding cholera toxin. Science (80-.). 272, 1910-1913 (1996).
41. Schroeder, C. M. et al. Antimicrobial resistance of Escherichia coli O157 isolated from humans, cattle, swine, and food. Appl. Environ. Microbiol. 68, 576-581 (2002).
42. Rafiq Ahmed, Cheryl Bopp, Al Borczyk, S. K. Phage-Typing Scheme for Escherichia coli O157:H7. J. Infect. Dis. 155, 806-809 (1987).
43. World Health Organization. Food safety. (2020). Available at: https://www.who.int/news-room/fact-sheets/detail/food-safety. (Accessed: 24th August 2021)
44. Tian, L., He, L., Jackson, K., Mahabir, R. & Hosseinidoust, Z. Bacteria repellent protein hydrogel decorated with tunable, isotropic, nano-on-micro hierarchical microbump array. Chem. Commun. 57, 10883-10886 (2021).
45. McCuen, R. Molecular cloning: a laboratory manual. Cold spring Harb. Lab. Press 186, 182-183 (1990).
46. Kropinski, A. M., Mazzocco, A., Waddell, T. E., Lingohr, E. & Johnson, R. P. Enumeration of bacteriophages by double agar overlay plaque assay. Bacteriophages: Methods and Protocols, Volume 1: Isolation, Characterization, and Interactions 501, (2009).
47. March, S. B. & Ratnam, S. Sorbitol-MacConkey medium for detection of Escherichia coli O157:H7 associated with hemorrhagic colitis. J. Clin. Microbiol. 23, 869-872 (1986).
48. Kifelew, L. G. et al. Efficacy of phage cocktail AB-SA01 therapy in diabetic mouse wound infections caused by multidrug-resistant Staphylococcus aureus. BMC Microbiol. 20, 1-10 (2020).
49. Guenther, S. & Loessner, M. J. Bacteriophage biocontrol of Listeria monocytogenes on soft ripened white mold and red-smear cheeses. Bacteriophage 1, 94-100 (2011).
50. Perera. M. N., Abuladze. T., Li. M., Woolston, J. & Sulakvelidze. A. Bacteriophage cocktail significantly reduces or eliminates Listeria monocytogenes contamination on lettuce. apples, cheese, smoked salmon and frozen foods. Food Microbiol. 52. 42-48 (2015).

1. A high-throughput method of synthesizing biofunctional microparticles, the method comprising:
a) casting biofunctional microparticle precursors onto a microporous template to form microparticles, wherein the template comprises a removable film; and
b) removing the film to liberate the microparticles.

2. The method of claim 1, wherein the template and/or film comprises polystyrene, polyvinyl chloride, polycarbonate, polyimide, polyvinyl chloride, polyvinyl butyral, or combinations thereof.

3. The method of claim 1 or 2, wherein the microporous template is prepared by the breath figure method, micro-templating, or photolithography.

4. The method of any one of claims 1 to 3, wherein the size range of micropores and resulting microparticles is between about 0.1 μm and about 999.9 μm.

5. The method of any one of claims 1 to 4, wherein the micropores and resulting microparticles are spherical, semi-spherical, cylindrical, or spindle-shaped.

6. The method of any one of claims 1 to 5, wherein the biofunctional microparticle precursors comprise biological materials, synthetic materials, or combinations thereof.

7. The method of claim 6, wherein the biological materials comprise proteins, peptides, enzymes, antibodies, nucleic acids, viruses, phages, prokaryotic cells, eukaryotic cells, or combinations thereof.

8. The method of claim 7, wherein the phages comprise unmodified phages (wild type), chemically-modified phages, genetically-modified phages, or combinations thereof.

9. The method of any one of claims 1 to 8, wherein the biofunctional microparticle precursors comprise additives.

10. The method of claim 9, wherein the additives comprise chemical or physical crosslinkers, nanoparticles, phages, antibiotics, proteins, peptides, nucleic acids, viruses, polymers, or combinations thereof.

11. The method of any one of claims 1 to 10, wherein the biofunctional microparticle precursors are self-crosslinked or crosslinked with one or more physical or chemical crosslinkers.

12. The method of claim 10 or 11, wherein the crosslinker comprises glutaraldehyde, 1-Ethyl-3-(3-dimethylaminopropyl) carbodiimide, gold nanoparticles, polymeric crosslinkers, or combinations thereof.

13. The method of any one of claims 1 to 12, wherein the microparticles are prepared in a suspension, solid patch, or powder.

14. The method of claims 13, wherein the microparticle suspension comprises microgels.

15. The method of claim 14, wherein the microgels comprise phage microgels and a fluid.

16. The method of claim 15, wherein the fluid comprises water, a buffer solution, phosphate buffered saline (PBS), a beverage, a medicine, or combinations thereof.

17. The method of any one of claims 13 to 16, wherein the microparticle suspension is for delivery in a spray, a suspension, by nebulization, or by injection.

18. Biofunctional microparticles made by the method of any one of claims 1 to 17.

19. A phage-built microgel made by the method of any one of claims 1 to 17.

20. A sprayable microgel made by the method of any one of claims 1 to 17.

21. A method of controlling bacteria, the method comprising applying the spray of claim 20 to a bacteria-susceptible surface.

22. The method of claim 21, wherein the surface comprises a food product, food packaging, or food contact environment.

23. The method of claim 22, wherein the food contact environment comprises a food packaging facility.

24. Use of the sprayable microgel of claim 20 for controlling bacteria in environmental, food chain, agricultural, pharmaceutical, medical, nutraceutical, textile, cosmetic, household, healthcare applications, or combinations thereof.
